(19) United States
(12) Patent Application Publication
(10) Pub. No.: US 2005/0245802 A1
(43) Pub. Date: Nov. 3, 2005

(54) Protective cover for ophthalmological tonometers and production method

(75) Inventor: Hartmut Kanngiesser, Zurich (CH)

Correspondence Address: OLIFF & BERRIDGE, PLC, P.O. BOX 19928, ALEXANDRIA, VA, 22320, US

(73) Assignee: SMT SWISS MICROTECHNOLOGY AG, Port (CH)

(21) Appl. No.: 11/107,936

(22) Filed: Apr. 18, 2005

(30) Foreign Application Priority Data
Apr. 29, 2004 (EP) 04 405 266.0

(51) Int. Cl. A61B 3/16

(52) U.S. Cl. 600/398

(57) ABSTRACT

A flexible protective cover (1) for ophthalmological tonometers which have a contact area for placing on an eye and a method for producing the protective cover (1) in one piece are proposed. The protective cover (1) has a flexible membrane (14) with a membrane thickness of less than 55 μm for covering the contact area. In the production of the protective cover (1), in a first step elastic material is injected into a cavity between an outer mould part and an inner mould part. In a second step, part of the protective cover (1) is formed as a membrane (14) for covering the contact area by the inner mould part and the outer mould part being moved towards each other in the region of the membrane (14) to be formed, so that a membrane (14) with a constant membrane thickness in the range from 30 μm to 50 μm is formed. The production method permits inexpensive production of flexible protective covers (1) in one piece with thin membranes (14) which do not impair eye-pressure measurements, or only slightly.

### TECHNICAL FIELD

The present invention relates to a protective cover for ophthalmological tonometers and to a method for producing the protective cover. The invention relates in particular to a protective cover for ophthalmological tonometers which have a contact area for placing on an eye, and to a method for producing the protective cover, a first step comprising that elastic material is injected into a cavity between an outer mould part and an inner mould part.

### PRIOR ART

In ophthalmology, tonometers are used for measuring the pressure of the eye. With tonometers, which have a contact area for placing on the eye, it is possible when the eye is touched by the contact area for lacrimal fluid, and with it also pathogens, to be transferred from one patient to another. Moreover, germs or contaminants which adhere to the contact area can be introduced into the eye during the measurement. To avoid this, the tonometer, in particular its contact area, is correspondingly cleaned or provided with a clean protective cover which is as sterile as possible. During eye examinations, eye-pressure measurements are performed on virtually every patient, the measurements generally taking a matter of just a few seconds. It should consequently also be possible for the preparation of the tonometers, in particular their measuring heads and contact areas, for the measurement to be accomplished as quickly as possible. Otherwise, the attendant doctor or optician must have a number of tonometers or measuring heads at his disposal, which he uses alternately. Generally, disposable protective covers are used, intended for use only once. In view of the large number of eye-pressure measurements and the protective covers used for them, it should be possible for the protective covers to be produced as inexpensively as possible. Moreover, the protective covers should be of such a kind that they impair the eye-pressure measurement as little as possible or not at all. In particular for tonometers which comprise pressure sensors, for example a tonometer according to EP 1250884, the protective covers for covering the contact area and the pressure sensor let into it should have membranes with wall thicknesses which do not impair the pressure measurement. In conventional injection-moulding processes it has been possible at best to produce protective covers with membranes which have a wall thickness of 70 μm, it being required for the injection to be performed centrally thereto in the membrane region. Both membranes with wall thicknesses of 70 μm and central injection points in the membrane region impair eye-pressure measurement by tonometers with pressure sensors to a substantial extent.

In the patent specification U.S. Pat. No. 5,343,861 there is a description of a protective cover formed in one piece, which is formed from transparent material, for example silicone, and can be applied to an applanation tip of a tonometer in order to avoid direct contact of the tip with a patient's eye. The protective cover according to U.S. Pat. No. 5,343,861 is firmly held on the tip by its elasticity, the protective cover preferably being of such a kind that a press fit with a corresponding part of the tip is brought about. The protective cover according to U.S. Pat. No. 5,343,861 has at its tip a circular part with a thickness in the range from 25 μm to 300 μm

### SUMMARY OF THE INVENTION

It is an object of the present invention to propose a novel protective cover for ophthalmological tonometers and a novel method for producing the protective cover.

It is, in particular, an object of the present invention to propose a protective cover for ophthalmological tonometers which have a contact area for placing on an eye and a method for producing the protective cover, it being intended that the protective cover can be produced as inexpensively as possible and that the eye-pressure measurement is impaired as little as possible by the protective cover, or not at all.

The aforementioned aims are achieved by the present invention in particular by the protective cover for ophthalmological tonometers which have a contact area for placing on an eye having for covering the contact area a flexible membrane with a membrane thickness of less than 55 μm, preferably a constant membrane thickness in the range from 30 μm to 50 μm, and by the protective cover being produced in one piece. The production of the protective cover in one piece with such a thin membrane has the effect on the one hand of dispensing with technically complex method steps for joining the membrane together with one or more carrier parts and on the other hand that the measurements carried out by means of a pressure sensor of the tonometer are impaired only slightly by the membrane, or not at all.

According to the invention, the protective cover has an opening and a surround enclosing the opening. The surround preferably forms an inner space tapering conically from the opening to the membrane and has, according to the invention, an adhering region facing the inner space. This form of the protective cover makes it possible for a conically tapering (substantially frustoconical) measuring head tip of the tonometer to be inserted through the opening and the measuring head tip to be received in the inner space, the contact area being covered by the membrane. The adhering region facing the inner space has the effect that the protective cover adheres to the measuring tip of the tonometer in the applied state.

The surround is preferably produced from an elastomer, for example from silicone, and the adhering region has a smooth surface and is arranged in a region that runs around the opening. The use of an elastomer for the surround on the one hand provides the extensibility and elasticity of the surround and on the other hand allows the adhesive effect of the surface of the protective cover to be controlled by structuring of the said surface. A roughened surface provided with an erosion structure has a small adhesive effect, while a polished, smooth surface has a great adhesive effect. The fact that the adhering region is arranged in the region which is facing the inner space and runs around the opening means that the protective cover remains adhering to the inserted measuring head tip in the peripheral region which is facing the inner space and adjacent to the opening. The flexible surround can be easily stretched thereby and in this way transmit a tensile effect to the membrane.

The membrane is preferably produced from an elastomer, for example from silicone, and has a smooth surface, which adheres on the contact area in the state of the protective cover in which it is applied to the measuring head tip. The smooth surface has the result that the membrane produced from an elastomer is provided with an adhesive effect as described above. If air which is enclosed between the membrane and the contact area is forced out of the region between the membrane and the contact area when the protective cover is applied, for example by means of a stamp, the air forced out can be prevented from re-entering this region by the membrane adhering to the contact area and by the above-mentioned tensile force. The forcing out of air from the region between the membrane and the contact area prevents the measurement of the eye pressure being falsified by inclusions of air.

In an embodiment, the membrane is concavely formed, viewed from outside the protective cover. The concave form of the membrane permits optimum adaptation to measuring head tips with a concavely formed contact area.

In an embodiment, the surround has channels facing the inner space, which extend from the membrane to the opening. These channels permit the removal of air from the region between the membrane and the contact area. In particular, the removal of forced-out air is made possible when the measuring head tip is inserted into the protective cover and the surround is bearing against the measuring head tip.

In an embodiment, the surround has a protruding collar, running around the opening, and the aforementioned adhering region is arranged on the part of the collar facing the inner space. The collar has the advantage that, in the state of the protective cover in which it is applied to the measuring head tip, it can catch liquids, for example lacrimal fluid, which run along the measuring head tip when the measuring head tip is placed on an eye. This prevents a grip that is connected to the measuring head tip from becoming slippery. Moreover, the protective cover on the collar can be manually removed from the measuring head.

The membrane preferably has a Shore hardness of less than 30 to 40. This permits uniform application of the protective cover to the measuring head tip, in particular to the contact area.

Apart from the protective cover, the present invention also relates to a method for producing the protective cover in one piece. In a first step, elastic material is injected into a cavity between an outer mould part and an inner mould part. According to the invention, in a second step, part of the protective cover is formed as a membrane for covering the contact area, the inner mould part and the outer mould part being moved towards each other in the region of the membrane to be formed, so that a membrane thickness of less than 55 μm is formed. The forming that follows the injection-moulding operation and is brought about by moving the inner and outer mould parts towards each other allows flexible protective covers with membrane thicknesses below 70 μm to be produced in one part and therefore inexpensively.

### BRIEF DESCRIPTION OF THE DRAWINGS

An implementation of the present invention is described below on the basis of an example. The example of the implementation is illustrated by the following accompanying figures:

FIG. 1 shows a cross section which schematically illustrates a flexible protective cover.

FIG. 2 shows a view which schematically illustrates the flexible protective cover with indicated channels.

FIG. 3 shows a plan view which schematically illustrates the flexible protective cover with the channels.

FIG. 4a shows a cross section which schematically illustrates two mould parts of a hot runner mould in the position of the injection operation.

FIG. 4b shows a cross section which schematically illustrates the two mould parts of the hot runner mould in the nominal position of the forming operation.

FIG. 5 shows a cross section which schematically illustrates a measuring head tip of an ophthalmological instrument part with the protective cover applied to it.

FIG. 6 shows a cross section which schematically illustrates the measuring head tip with a concave contact area and the protective cover applied to it.

### WAYS OF IMPLEMENTING THE INVENTION

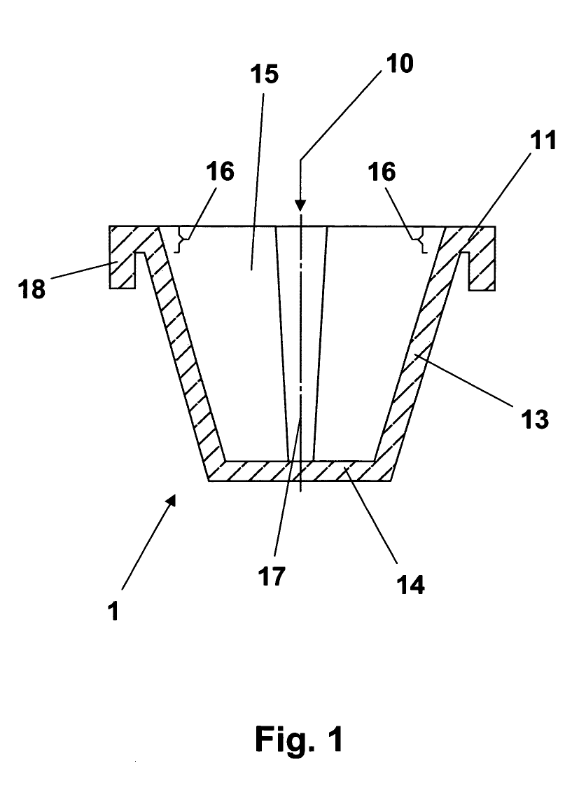

In FIGS. 1, 2, 3, 4a, 4b, 5 and 6, corresponding, identical components are designated by the same reference numerals.

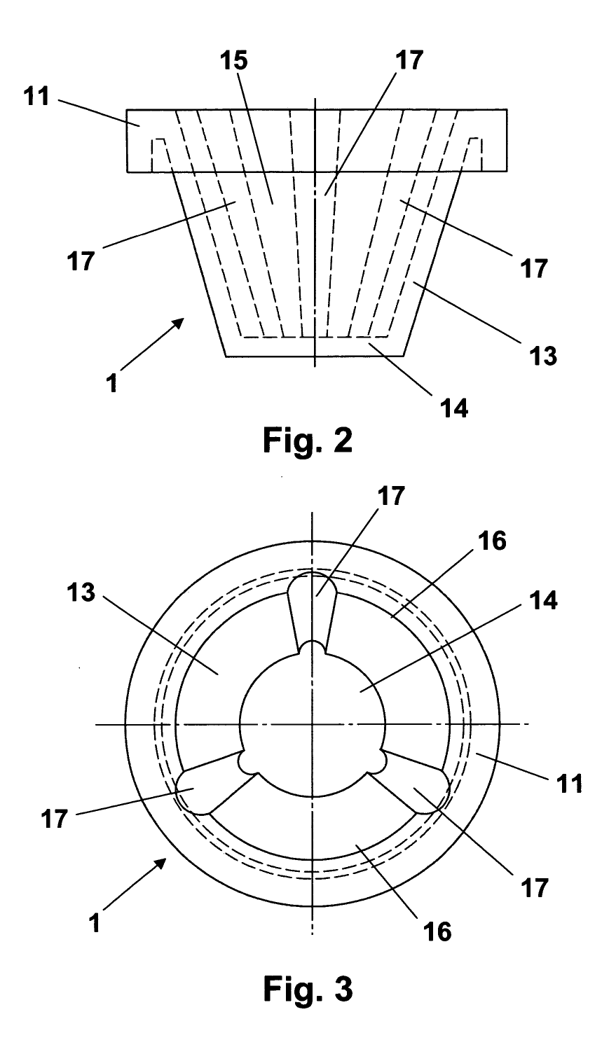

In FIGS. 1, 2, 3, 4a, 4b, 5 and 6, the reference numeral 1 designates a flexible protective cover made of an elastomer, for example silicone, polyurethane, plasticized PVC or rubber. As can be seen from FIG. 1, the protective cover 1 is formed in a cup-shaped manner and has a circular base formed as a membrane 14 and a peripheral surround 13, connected to the membrane 14. The surround 13 comprises a peripheral collar 11, which has a substantially U-shaped profile, which is formed in a round or angular manner. The collar 11 forms a periphery, which runs around the opening 10 of the protective cover 1 and protrudes from the surround 13 in the direction facing away from the inner space 15. The diameter of the opening 10 is, for example, 9.8 mm. The membrane 14 has a highly polished, smooth surface, preferably on both sides. The membrane 14 has a constant thickness in the range from 40 μm to 50 μm, for example 45 μm. The diameter of the membrane 14 is, for example, 8 mm. The inner space 15 of the protective cover 1 that is formed by the membrane 14 and the surround 13 tapers conically from the collar 11 to the membrane 14 and, in the case of a planar membrane 14, is substantially frustoconical. As schematically represented in FIGS. 2 and 3, the inner region of the surround 13 facing the inner space 15 has a number of channels 17, which respectively extend in a straight line from the membrane 14 to the collar 11. Although it is not represented in FIGS. 2 and 3, the surround 13 has, for example, eight symmetrically arranged channels 17. The surround 13 has, for example, a wall thickness which tapers continuously from 0.5 mm in the region of the collar 11 to 0.1 mm in the region adjacent to the membrane 14. The transition from the surround 13 to the membrane 14 is, for example, rounded, for example with an inner radius of 0.3 mm and an outer radius of 0.35 mm. The protective cover 1 has a height from the membrane 14 to its opening 10 of, for example,
10 mm.

The inner region of the collar 11 facing the inner space 15 has a highly polished, smooth adhering region 16. The adhering region 16 has, for example, peripherally a width of 0.5 to 2 mm. Apart from at the aforementioned locations, that is the two sides of the membrane 14 and the adhering region 16, the protective cover 1 has a surface roughened by erosion structures.

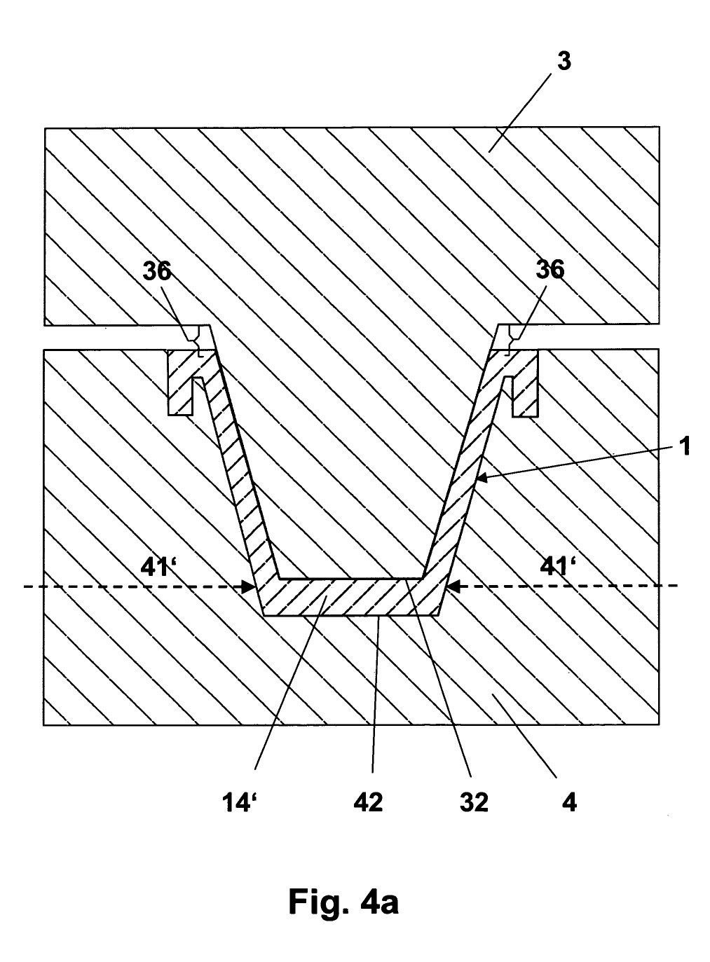

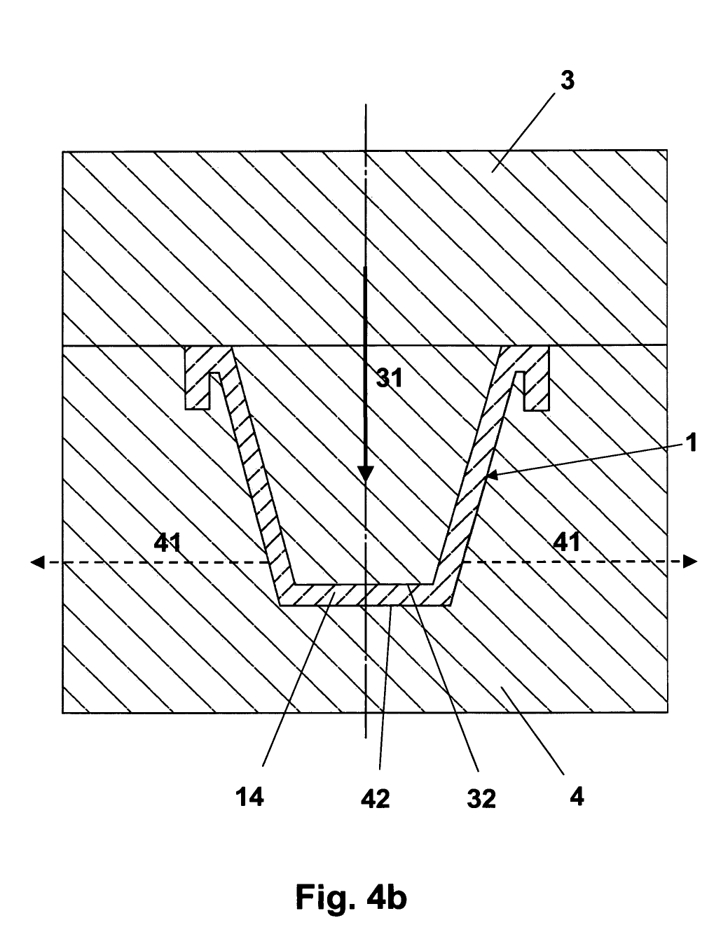

The protective cover 1 is produced in one piece by means of a hot runner mould. The protective cover 1 is produced in one piece by the injection-moulding process with subsequent forming. The surface finish of the protective cover 1 is determined by corresponding surface structures of the mould parts 3, 4 used (see FIG. 4a) of the hot runner mould. The corresponding surface regions 32 and 42, and 36, respectively, of the mould parts 3, 4 for forming the membrane 14 and the adhering region 16 are highly polished. As schematically represented in FIG. 4a by the arrows 41′, in a first step, the liquid elastic material is injected into the cavity between the mould parts 3, 4 in the membrane region. At the moment of injection, the core represented by the (inner) mould part 3 inside the (outer) mould part 4 is not located in the nominal position of the membrane thickness but, for example, about 300 μm ahead of that. In this injection position, the injection openings have free access to the membrane region. The formation of the collar 11 is not discussed in any more detail here, since the production of structures with such wall thicknesses is possible by conventional injection-moulding processes. After the injection of the elastic material, when the cavity between the mould parts 3, 4 is filled, the inner mould part 3 is brought into the nominal position, as indicated by the arrow 31, so that the surface regions 32 and 42 are at a distance of 50 μm to form a membrane thickness of 50 μm. As schematically represented in FIG. 4b by the arrows 41, the elastic material thereby forced out flows away. The connecting points produced as a result, located outside the membrane region, are mechanically removed after the curing of the elastic material. The forcing out of excess material in the collar region is not discussed here.

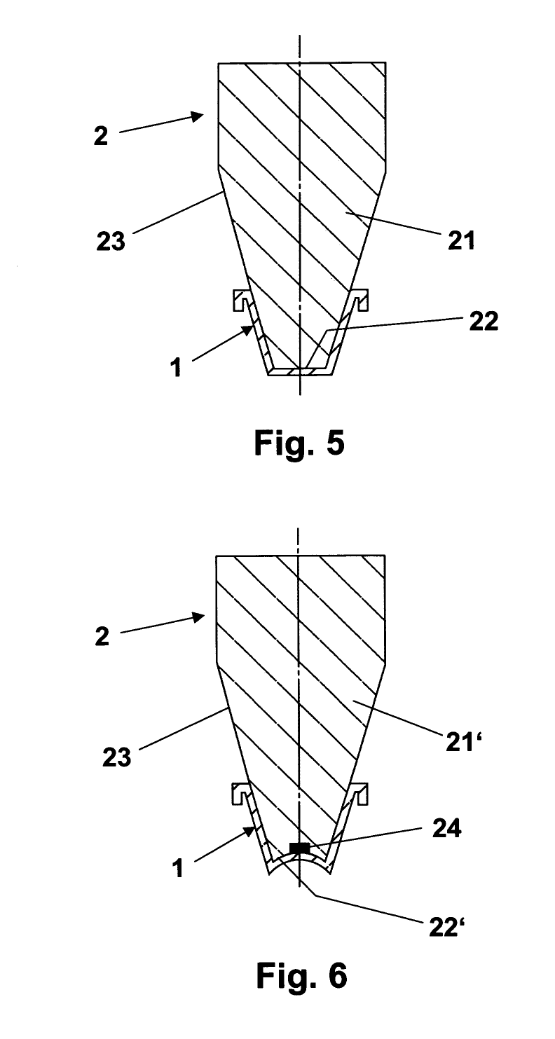

To apply the protective cover 1 to the measuring head tip 21 of the ophthalmological instrument part 2, the measuring head tip 21 is inserted through the opening 10 into the inner space 15 of the sterile protective cover 1 (FIGS. 5 and 6). During insertion, the side of the membrane 14 facing the inner space 15 comes into contact with the contact area 22 of the measuring head tip 21. By continuing the inserting movement, the outer region 23 of the measuring head tip 21 is pressed onto the adhering region 16 of the collar 11, whereby an adherence of the adhering region 16 to the outer region 23 of the measuring head tip 21 is brought about. Air which is trapped between the membrane 14 and the contact area 22 can be forced out for example by means of a convex, compressible stamp and be carried away over the outer region 23 of the measuring head tip 21, in particular via the channels 17. The protective cover 1 adheres both with the adhering region 16 to the outer region 23 of the measuring head tip 21 and with the membrane 14 to the contact area 22 of the measuring head tip 21, as represented in FIGS. 5 and 6. The protective cover 1 can be discarded or recycled after use.

As represented in FIG. 6, the proposed protective cover 1 is suitable not only for measuring heads 21 with planar contact areas 22, but also for concave (or convex) contact areas 22′. In the case of concave or convex contact areas, however, the radius of curvature of the pressure-exerting region of a stamp used for forcing out the air should be smaller than the radius of curvature of the contact area 22′. FIG. 6 shows in particular an example in which the protective cover 1 has been applied to a tip 21′ of a contour tonometer according to EP 1250884, which has a concave contact area 22′, into which a pressure sensor 24 with the same contour as the concave contact area 22′ is set.

Finally, it should be mentioned that the proposed protective cover 1 can be used even in the case of contact areas 22 which have surfaces with steps, offsets and/or gaps, if the latter may be spanned by a continuous curved shape without impairment of the measurement and in the stepped region air may be trapped between the protective cover 1 and the contact area 22.

Appendix to Search Report for European Patent Application No. EP 04 40 5266

This appendix gives the members of the family of patents relating to the patent documents cited in the above-identified search report.

These members were contained in the database of the European Patent Office as of 21-09-2004

The information supplied is for information only and the European Patent Office does not incur any liability thereunder.

| Patent Document | | | |
|---|---|---|---|
| Cited in Search | Publication | Member(s) of | Publication |
| Report | Date | Patent Family | Date |
| U.S. Pat. No. | 06-09-1994 | U.S. Pat. No. | 19-05-1992 |
| 5343861 A | | 5113863 A | |
| U.S. Pat. No. | 08-05-1990 | NONE | |
| 4922914 A | | | |
| U.S. Pat. No. | 18-02-1992 | AT 114808 T | 15-12-1994 |
| 5088834 A | | | |
| | | DE 69105408 D1 | 12-01-1995 |
| | | DE 472490 T1 | 15-10-1992 |
| | | DK 472490 T3 | 01-05-1955 |
| | | EP 0472490 A1 | 26-02-1992 |
| | | ES 2031058 T1 | 01-12-1992 |
| | | JP 1911967 C | 09-03-1995 |
| | | JP 4341236 A | 27-11-1992 |
| | | JP 6042872 B | 08-06-1994 |
| WO 0239892 A | 23-05-2002 | EP 1208792 A1 | 29-05-2002 |
| | | AU 1203802 A | 27-05-2002 |
| | | BR 0115405 A | 17-02-2004 |
| | | CA 2427908 A1 | 23-05-2002 |
| | | WO 0239892 A1 | 23-05-2002 |
| | | CN 1474667 T | 11-02-2004 |
| | | EP 1335664 A1 | 20-08-2003 |
| | | NO 20032223 A | 16-05-2003 |
| | | U.S. Pat. No. | 26-02-2004 |
| | | 2004036839 A1 | |
| U.S. Pat. No. | 07-06-1994 | NONE | |
| 5318029 A | | | |

For all information concerned with this Annex: See Official Journal of European Patent Office No. 12/82.

1. A protective cover for ophthalmological tonometers which have a contact area for placing on an eye, the protective cover being in one piece, the protective cover comprising a flexible membrane for covering the contact area which membrane has a membrane thickness of less than 55 μm, and the protective cover having an opening and a surround enclosing the opening, which surround forms an inner space, wherein the surround has an adhering region facing the inner space.

2. The protective cover according to claim 1, wherein the surround forms an inner space tapering conically from the opening to the membrane, and the adhering region is arranged in a partial region of the surround that runs around the opening and is adjacent to the opening.

3. The protective cover according to claim 1, wherein the surround is produced from an elastomer, the adhering region has a smooth surface, and the region of the surround facing the inner space that lies outside the partial region in which the adhering region is arranged has a roughened surface.

4. The protective cover according to claim 1, wherein the membrane is produced from an elastomer, and the membrane has a smooth surface, which surface adheres on the contact area in the state of the protective cover in which it is applied to the tonometer.

5. The protective cover according to claim 1, wherein the membrane is concavely formed, viewed from outside the protective cover.

6. The protective cover according to claim 1, wherein the membrane has a constant membrane thickness in the range from 30 μm to 50 μm.

7. The protective cover according to claim 1, wherein the surround has channels facing the inner space, which extend from the membrane to the opening.

8. The protective cover according to claim 2, wherein the surround has a protruding collar, running around the opening, and the said adhering region is arranged on a part of the collar facing the inner space.

9. The protective cover according to claim 1, wherein the membrane has a Shore hardness of less than 30 to 40.

10. A method for producing in one piece a protective cover for ophthalmological tonometers which have a contact area for placing on an eye, the method comprising:
a first step of injecting elastic material into a cavity between an outer mould part and an inner mould part, and
a second step of forming part of the protective cover as a membrane for covering the contact area, the inner mould part and the outer mould part being moved towards each other in the region of the membrane to be formed, so that a membrane thickness of less than 55 μm is formed.

11. The method according to claim 10, wherein the inner mould part and the outer mould part are formed in such a way that the protective cover according to one of claims 1 to 9 is formed.
